(19) United States
(12) Patent Application Publication
(10) Pub. No.: US 2026/0126433 A1
(43) Pub. Date: May 7, 2026

(54) Cell-Free Transcriptional Electrochemical Biosensors for Detecting Molecular Analytes, and Method Thereof

(71) Applicants: Francesco Ricci, Roma (RM) (IT); Simona Ranallo, Roma (RM) (IT); Sara Bracaglia, Frosinone (FR) (IT); Consorzio Interuniversitario Istituto Nazionale Di Biostrutture E Biosistemi, Roma (RM) (IT)

(72) Inventors: Francesco Ricci, Roma (IT); Simona Ranallo, Roma (IT); Sara Bracaglia, Frosinone (IT)

(21) Appl. No.: 19/117,512

(22) PCT Filed: Oct. 17, 2023
(86) PCT No.: PCT/IB2023/060468

§ 371 Date: Apr. 1, 2025

(30) Foreign Application Priority Data
Oct. 18, 2022 (IT) 102022000021390

(51) Int. Cl. G01N 33/53; G01N 27/327; G01N 33/543; G01N 33/553; G01N 33/68

(52) U.S. Cl. CPC G01N 33/5308; G01N 27/3277; G01N 33/5438; G01N 33/553; G01N 33/6803; G01N 33/6854

(57) ABSTRACT

The present invention relates to a cell-free transcriptional electrochemical biosensor and to the use of the same for detecting specific molecular analytes, such as specific antibodies, proteins, small molecules, nucleic acids, and derivatives thereof, in complex arrays of biological samples, such as plasma, serum, blood, saliva, sweat, and the like, wherein said biosensor is based on the activation of the transcription of a specific RNA strand, induced by recognition with the analyte. The invention further relates to a method for the detection of specific molecular analytes in complex arrays of biological samples, said method being based on the use of said cell-free transcriptional electrochemical biosensor.

### TECHNICAL FIELD OF THE INVENTION

The present invention relates to a cell-free transcriptional electrochemical biosensor and to the use of the same for detecting specific molecular analytes, such as, for example, specific antibodies, proteins, small molecules, nucleic acids, or derivatives thereof, in complex arrays of biological samples, such as, for example, plasma, serum, blood, saliva, sweat, and the like, wherein said biosensor is based on the activation of the transcription of a specific RNA strand, induced by recognition with the analyte.

The invention further relates to a method for the detection of specific molecular analytes in complex arrays of biological samples, said method being based on the use of said cell-free transcriptional electrochemical biosensor.

### DESCRIPTION OF THE KNOWN ART

Antibody detection is important in several clinical contexts, in that it provides useful information on present and past infections, and can also provide information about the clinical outcomes when treating and monitoring autoimmune diseases and cancer.

Reference assays and methods, also called gold standards, which are commonly used for detecting antibodies and proteins, are selectable, for example, among the following: a) ELISA (Enzyme Linked Immunosorbent Assay) and b) LFT (Lateral Flow Test, or “lateral flow immunochromatographic assay”, or “rapid test”). ELISA assays are very sensitive, and are widely used for measuring antibodies, antigens and clinically relevant chemical substances. However, they are based on a multi-phase procedure, the execution of which may take a long time, sometimes longer than actually tolerable. Moreover, ELISA tests require many reagents, and this often makes them very costly. On the other hand, lateral flow immunological tests (LFT) are used in so-called point-of-care diagnostics because they are cheap and fast. However, they can only provide qualitative or semi-quantitative results.

By way of example, WO 2017/147486 describes the electrochemical detection of DNA amplification sequences, thanks to the use of a simple disposable electrochemical sensor suitable for measuring a DNA/RNA sequence by using methylene blue. The subject matter of WO 2017/147486 is not an example of cell-free technology.

In recent times, the so-called “synthetic biology” has been proposed as an alternative technique which can be used in order to overcome the above-mentioned limitations. Synthetic biology is based on a biomolecular engineering approach for in-vitro reproduction of processes that would otherwise only occur within cells. Actually, there is an ever increasing need for expanding the medical monitoring and diagnostics of human diseases and, from this point of view, the devices of synthetic biology might provide diagnostic methods with new potentialities, e.g. by creating sensors with new functions, thereby broadening the range of testable analytes (or targets) and improving the sensitivity and specificity of the same. The so-called cell-free biosensors utilize several biological components, such as, for example, nucleic acids (DNA) and proteins (enzymes). DNA acts as a template for the production of specific RNA sequences, while enzymes (e. g. a polymerase) catalyze the process of formation of RNA (transcription) and/or of other proteins (translation). In cell-free biosensors, the recognition of the target analyte by a DNA sequence induces the transcription or the expression of the final biomolecule (called output biomolecule; for example, a RNA or a protein). In recent years, cell-free biosensors have been developed which can detect specific sequences of messenger RNA (mRNA), small molecules and proteins, including antibodies (by the way, also due to the researches by the present inventors)⁽¹⁻⁶⁾.

Recently, Patino Diaz Aitor et al. have proposed a different approach that exploits a cell-free transcription system for detecting specific antibodies, as described in their article entitled: “Programmable cell-free transcriptional switches for antibodies detection”, Journal of The American Chemical Society, vol. 144, n. 13, 22 Mar. 2022, pp. 5820-5826 (cited herein for reference and available in its entirety at the address https://pubs.acs.org/doi/10.1021/jacs.1c11706). This article describes a programmable transcriptional switch based on a gene circuit designed to adopt a stem-loop conformation that prevents the transcription of an aptamer to a RNA capable of promoting a fluorescence signal (so called light-up signal), because the promoter region (so called promoter) is hidden inside this stem-loop structure and cannot be recognized by the RNA polymerase. In order to obtain a change in the conformation of the gene circuit induced by the antibody, two DNA strands conjugated with the specific antigen are used which, following the formation of a divalent bond with a target antibody, are brought into close mutual proximity (are co-located) and can hybridize to form a functional bimolecular complex. This complex induces a reaction of displacement of the DNA strand, such that the gene circuit assumes a linear conformation and thus makes the promoter sequence accessible, which can then be recognized by the RNA polymerase. The transcriptional switch is thus activated and, in the presence of a RNA polymerase and nucleotides, can induce the transcription of a fluorescent (light-up) RNA aptamer, which acts as a reporter, which can be measured by fluorescence (as indicated, for example, in the accompanying FIG. 1c). Patino-Diaz describes as an indispensable characteristic the achievement of a conformational change in response to the formation of the bond of the target protein/antibody. This fact is absolutely not required in the present invention.

### Drawbacks of the Known Art

### Drawbacks of the Methods for Antibody Detection Currently in Use

As previously described, notwithstanding their high sensitivity, the current gold standards (e.g., ELISA) for detecting antibodies and proteins are based on multi-step processes that are rather complex as a whole, since they require many washing steps and large amounts of reagents, so that undesired delays may occur in patient treatments. Unlike such tests, lateral flow tests (LFT) have essentially a single-step format and require no washing steps; however, they can usually provide qualitative results only, and cannot therefore be used in applications that require exact quantification. Moreover, the electrochemical sensors commonly employed for antibody detection are versatile, sensitive and rapid, but their response cannot be improved through the use of an enzyme reaction.

### Drawbacks of the Cell-Free Detection Methods Currently in Use

Cell-free biosensors can be used for meeting the increasing demand for novel diagnostic and analytic methods and tools with enhanced performance. Most of these sensors adopt optical methods in order to detect the transcribed or expressed fluorescent final biomolecule. While these sensors offer several advantages in terms of versatility and sensitivity, they however remain tied to the typical limitations of optical methods. This means that they give low performance in complex sample arrays (such as, for example, blood serum, whole blood, etc.) . Also, they cannot be adapted to a suitable low-cost, portable instrumentation. Just a few cell-free biosensors for the detection of nucleic acids and small molecules have been reported for use in an electrochemical detection method,^{(7,8)} which is much more suited for the point-of-care diagnosis. To the present inventors'knowledge, however, such strategies must necessarily use both transcription and expression systems, which are more difficult to control and imply a higher total cost per assay.

The development of suitable gene circuits which can be activated by a wide range of analytes and which can operate with transcription systems alone would be crucial in view of the practical use of the cell-free biosensors for facing many health challenges. In addition, due to the possible miniaturization of the surface of the sensor's electrode, the low cost of the sensor, and of the portable instrumentation, electrochemical detection might prove to be a detection mechanism of choice for the use of cell-free biosensors for the point-of-care diagnostic applications.

### Drawbacks of the Cell-Free Antibody Detection Method Published by the Present Inventors

The cell-free transcriptional switch for antibody detection reported in the above-mentioned article by the present inventors, annexed hereto for reference, has shown two main limitations:
1) it is based on optical detection, which, as previously mentioned, is the least appropriate technique for complex clinical samples (or arrays) (e.g., plasma, serum, blood, and the like);
2) it requires the rational design of a transcriptional switch that is non-active in the absence of the target antibody, and that is activated only when the target antibody binds to two specific antigen-conjugated DNA strands. For a person skilled in the art, such a rational design is neither trivial nor immediate to achieve, but it requires substantial experimental efforts and investigations to identify the best conditions under which a low background signal can be observed in the absence of the target antibody and a high signal can be observed in the presence of the target antibody.

### DESCRIPTION OF THE DRAWINGS

FIG. 1 discloses: (a) the general diagram of the process of activation of the transcriptional switch induced by the target antibody following the reaction of displacement of the DNA strand which causes the gene circuit to assume a linear configuration; (b) the general scheme of the process of reconstitution of the gene circuit induced by the target antibody; (c) the absorption and emission spectra of the light-up aptamer in the presence and absence of the target antibody; (d) the electrochemical detection of the transcribed RNA after hybridization with the redox probe immobilized on the surface of a screen-printed electrode in the presence and absence of the target antibody.

FIG. 2 discloses the general diagram of the cell-free electrochemical biosensor for antibody detection: (a) antibody-induced activation of the gene circuit. Two antigen-conjugated DNA strands can be co-located after binding to the target antibody, thus forming a bimolecular complex capable of hybridizing to the inactive gene circuit and of activating it by reconstitution of the promoter linear region; (b) in the presence of RNA polymerase and nucleotides, the gene circuit thus activated leads to the transcription of a final (output) RNA strand; (c) the transcribed final RNA strand can be detected by means of an electrochemical sensor formed of a screen-printed silver electrode on which a complementary redox probe has been previously immobilized. Hybridization of the transcribed RNA to this redox probe results in a decreased electrochemical signal, which can be measured using the known square-wave voltammetry (SWV) technique.

FIG. 3 discloses: (a) the general diagram of the cell-free electrochemical biosensor for detecting the target Anti-Dig antibody; (b) SWV voltammetric graphs obtained in the absence and presence of the Anti-Dig antibody; (c) signal percent-variation values obtained with increasing concentrations of the Anti-Dig antibody and (d) at saturating concentrations (300 nM) of Anti-Dig antibody and of non-specific antibodies, as well as in different control tests.

FIG. 4 discloses: (a) the general diagram of the cell-free electrochemical biosensor for detecting the target Anti-DNP antibody; (b) SWV voltammetric graphs obtained in the absence and presence of the Anti-DNP antibody; (c) signal percent-variation values obtained with increasing concentrations of the Anti-DNP antibody and (d) at saturating concentrations (300 nM) of Anti-DNP antibody and of non-specific antibodies, as well as in different control tests.

FIG. 5 discloses: (a) the general diagram of the cell-free electrochemical biosensor for detecting the target Anti-HA antibody; (b) SWV voltammetric graphs obtained in the absence and presence of the Anti-HA antibody; (c) signal percent-variation values obtained with increasing concentrations of the Anti-HA antibody; (d) quantification of the Anti-HA antibody, evaluated by means of serum samples fortified with different known concentrations of the Anti-HA antibody (15, 25, 32 and 45 nM).

FIG. 6 discloses: (a) the general diagram for simultaneously detecting the Anti-DNP and Anti-HA antibodies using two orthogonal cell-free electrochemical biosensors in the same solution. After the transcription of the reaction, the solution is placed on the surface of an electrode with two different working electrodes, each one containing the specific redox probe for each gene circuit; b) SWV voltammetric graphs obtained from different experiments conducted with a sample solution containing different combinations of both antibodies.

### SUMMARY OF THE INVENTION

It is the object of the present invention a method for the electrochemical detection, with high sensitivity and specificity, of molecular analytes, in particular selected from antibodies, proteins, small molecules, nucleic acids, and/or derivatives thereof, preferably antibodies, directly in complex sample arrays, such as, for example, plasma, serum, blood, or the like, which has proven to be particularly suitable for the point-of-care diagnostics. The approach is based on the use of antigen-conjugated programmable gene circuits which, following the recognition of a specific target analyte/antibody, trigger the in-vitro transcription of a specific RNA sequence that can subsequently be detected using a strand of a redox probe immobilized on a screen-printed electrode, e.g., a disposable one. Screen-printed electrodes (SPE) have a three-electrodes configuration including: a working electrode (WE) made of silver, a reference electrode (RE) made of silver, and a counter-electrode (CE) made of graphite. The applications of the method of the invention include, without being limited thereto, the electrochemical detection of antibodies/antigens/proteins by detecting a specific antibody-induced, in-vitro transcribed RNA sequence.

### Detection Principle

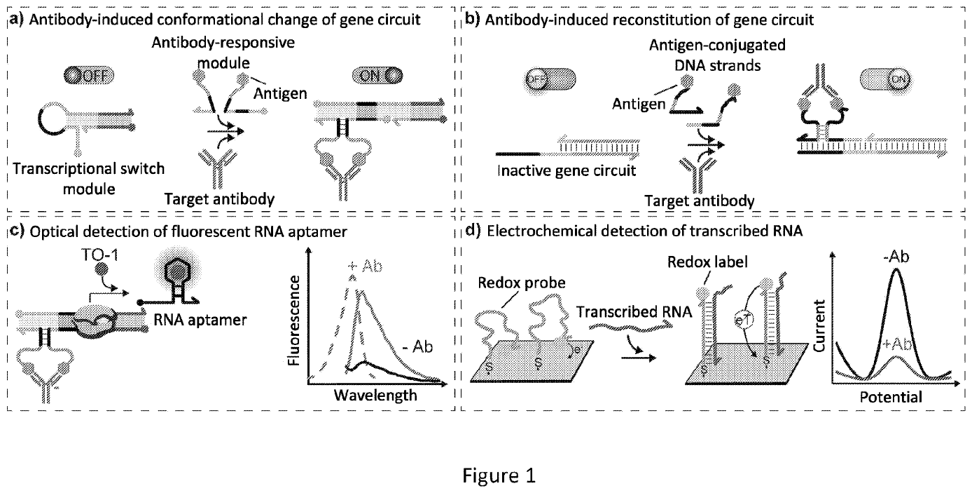

In the present invention, the cell-free biosensor is based on the use of DNA strands rationally designed so that the gene circuit contains the incomplete T7 promoter region (promoter) that impedes the transcription process by the T7 RNA polymerase (T7-RNAP, as described in the accompanying FIG. 1b) ). This method requires, in a first aspect, an appropriate design of the antigen-conjugated strands (i.e., the so-called input strands) that should hybridize to the inactive gene circuit, and then activate it only upon formation of a divalent bond with the target analyte/antibody. In order to achieve this result, it was necessary to optimize these antigen-conjugated input strands so that they were fulfilling two main conditions. Firstly, they had to be programmed in such a way that, at the adopted experimental concentrations, they could not hybridize to the inactive gene circuit, whereas when joined together into a bimolecular complex they would be able to hybridize efficiently thereto and activate the transcription. Secondly, under the adopted experimental conditions, they should remain separate, forming a stable bimolecular complex only upon co-location induced by the divalent bond with the target antibody. In the presence of RNAP and nucleotides, the gene circuit thus activated can transcribe a specific (output) RNA sequence, which can then be detected by an electrochemical instrumentation (as described in the accompanying FIG. 1d) ).

### DETAILED DESCRIPTION OF THE INVENTION

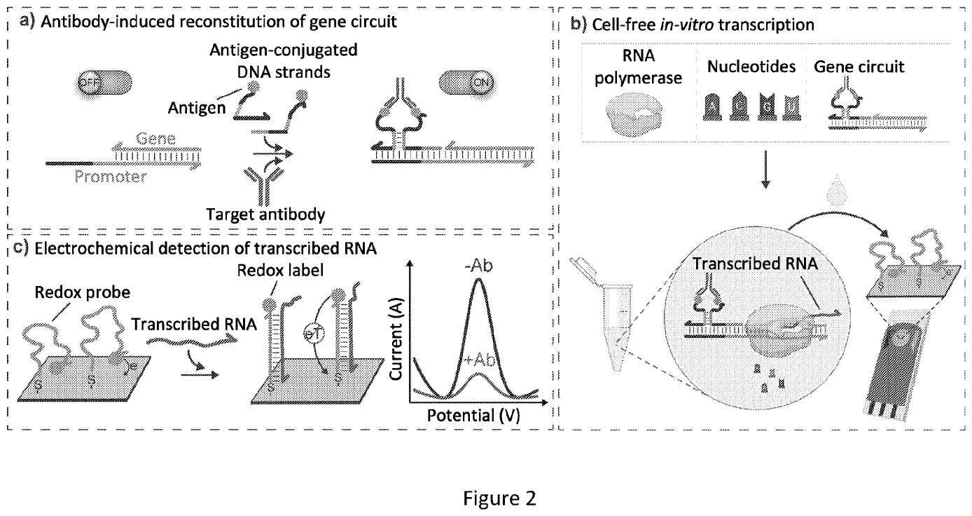

As briefly described above, the method of the present invention is based on the activation, induced by one or more target analytes, preferably antibodies, of an initially incomplete (i.e., inactive) DNA gene circuit, which, once activated, transcribes a final (output) RNA sequence by means of a RNA polymerase and suitable nucleotides. Said RNA sequence, thus transcribed, is in turn detected by the electrochemical biosensor of the present invention, which will be described below. The gene circuit is appropriately designed to contain the incomplete region of the T7 promoter, e.g., lacking the last 5 recognition bases, which effectively prevents the formation of the bond with the T7 RNA polymerase enzyme (T7-RNAP), and prevents the transcription of the final output RNA strand (as described in the accompanying FIG. 2A) ). In order to obtain the antibody-induced activation of said gene circuit, a pair of suitable antigen-conjugated (input) DNA strands were used, rationally designed to form a bimolecular complex only upon formation of a divalent bond between the target antibody and both antigens. This bimolecular complex can efficiently hybridize to the single-stranded portion of the gene circuit and reconstitute the complete sequence of the promoter (T7) region (as described in the accompanying FIG. 2A) ). In the presence of the T7-RNAP enzyme, and of the known necessary nucleotides, the gene circuit thus activated can be used as a template for the transcription of the final (output) RNA strand (as described in the accompanying FIG. 2b) ). The transcribed RNA sequence can be detected by an electrochemical biosensor formed of a screen-printed silver electrode (e.g., a disposable one) on which a redox probe complementary to the transcribed RNA sequence has been previously immobilized.

Said probe is marked at one end with a molecule of methylene blue and at the other end with a thiol group. This probe is designed to selectively bind to the transcribed RNA strand, leading to the formation of a stiffer double-stranded (duplex) complex, which reduces the efficiency with which the terminal redox label collides with the electrode's surface and transfers the electrons, thereby causing a reduction in the produced farad current. As is known, the hybridization of the RNA transcript that results in a decreased electrochemical signal can be measured, for example, by using the square-wave voltammetry (SWV) (as described in the accompanying FIG. 2c)).

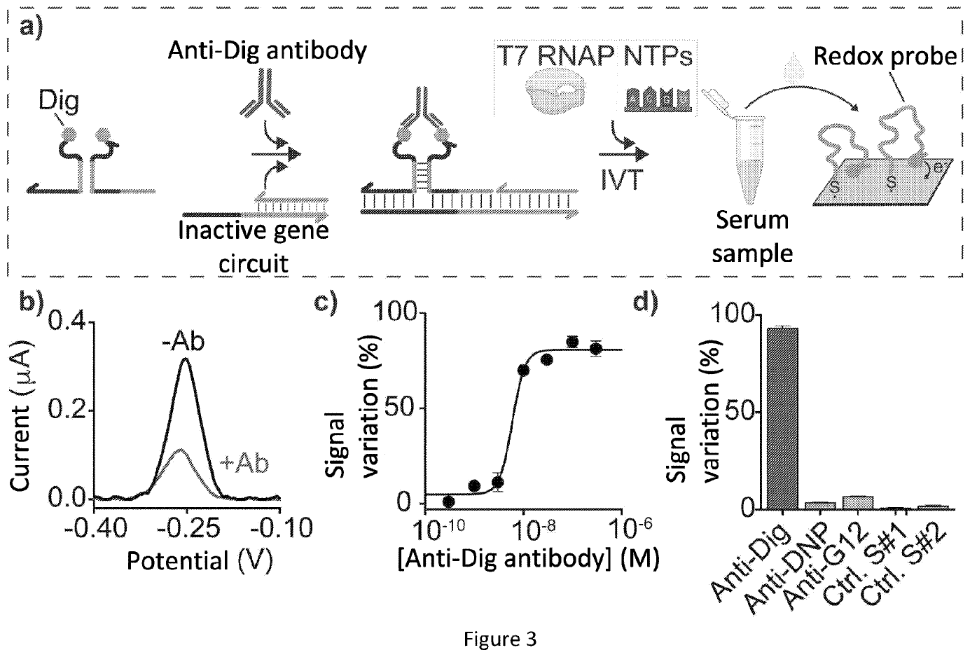

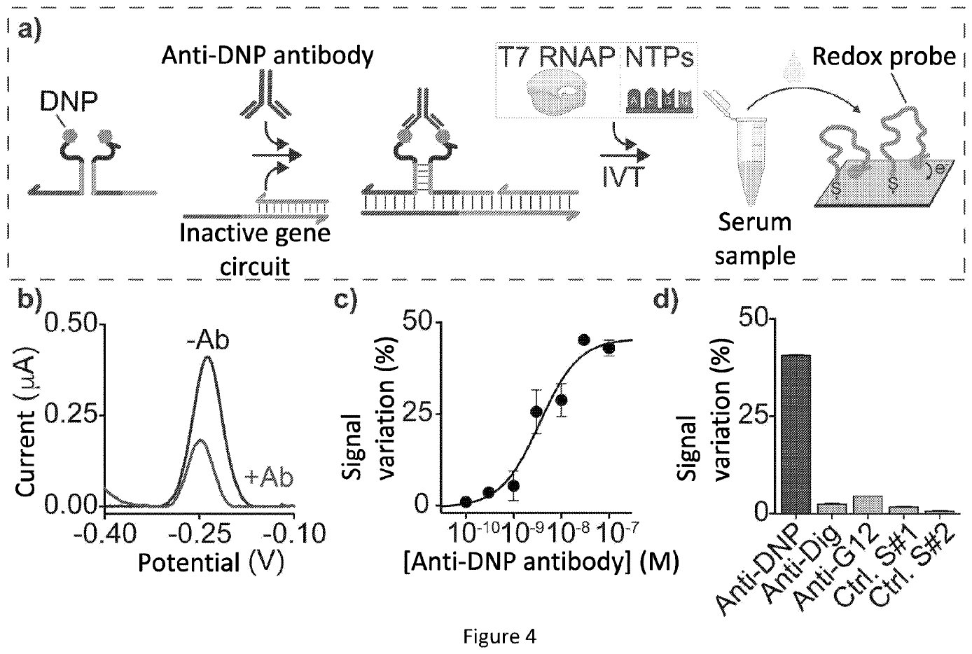

The RNA transcription can be activated by the target antibody binding to the conjugated strands, e.g., to the different recognition elements (antigens) digoxigenin (Dig) and/or dinitrophenol (DNP). Only after the formation of a divalent bond of the Anti-Dig and/or Anti-DNP antibodies, respectively, the two antigen-conjugated strands are joined together to form a bimolecular complex complementary to the single-stranded portion of the inactive gene circuit, thus permitting the transcription of the final RNA sequence (as described in the accompanying FIGS. 3A), 4A)). The electrochemical detection of these antibodies was demonstrated by placing a portion of the transcription reaction onto the surface of the screen-printed electrode (preferably, of the disposable type) to cause a farad current reduction only in the presence of the target antibody (as described in the accompanying FIGS. 3b), 4b) ). Using a titration of the Anti-Dig and Anti-DNP antibodies from 300 nanomolar to 300 picomolar, the sensors were characterized as a function of antibody concentration (as described in the accompanying FIGS. 3c), 4c)). The results show an increased signal variation with increasing concentrations of the target antibody. The method proved to be specific, because no signal variation was observed in the presence of non-specific antibodies or in the absence of either one of the two antigen-conjugated strands (as described in the accompanying FIGS. 3d), 4d)).

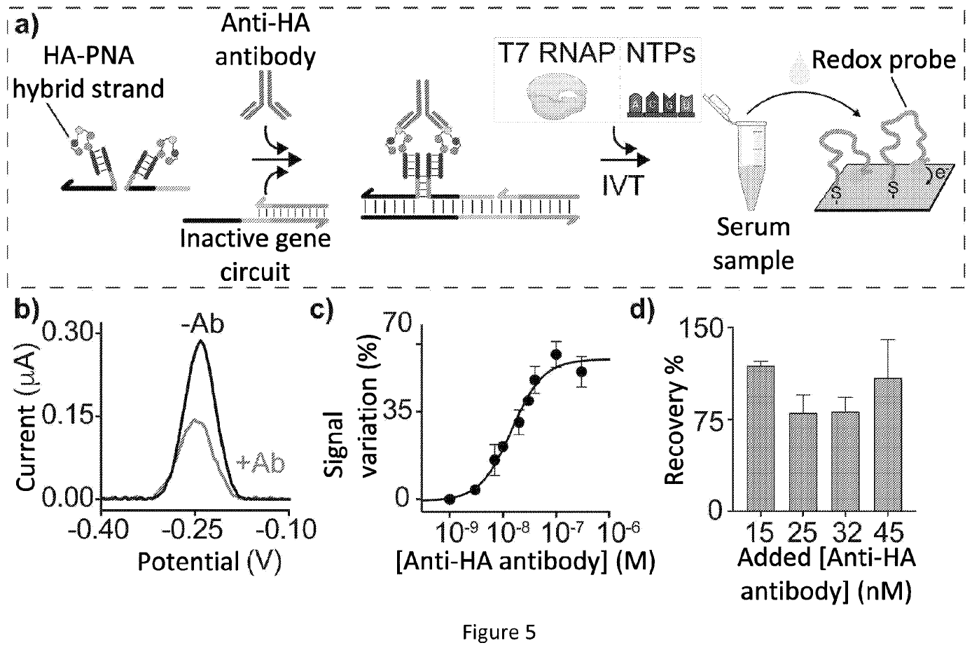

The method of the present invention can also use a modular approach, wherein synthetic oligonucleotides of peptide nucleic acid (PNA) are conjugated to the recognition element (the HA epitope) and hybridize to the complementary synthetic oligonucleotides (as described in the accompanying FIG. 5A)). With this modular approach, the method of the present invention is more easily applicable to more complex recognition elements (peptide epitopes, proteins) that require difficult and costly procedures for the conjugation to synthetic DNA strands. Just like its non-modular counterpart, the modular approach proved to be both sensitive and specific (as described in the accompanying FIG. 5b)-5c)). The recovery percentages of serum samples fortified with four different concentrations of Anti-HA were also evaluated, obtaining good precision with recovery percentages ranging from 80% to 119% (as described in the accompanying FIG. 5d)).

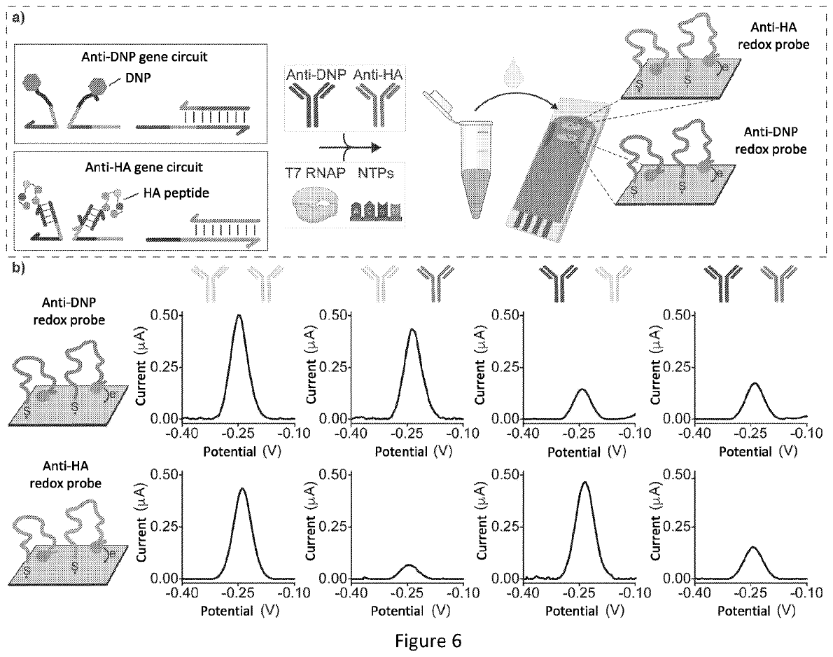

These methods can be implemented orthogonally in multiplex reactions to permit the detection of different antibodies in the same sample solution (as described in the accompanying FIG. 6A)). The antibody-controlled reactions can generate a current signal reduction only in the corresponding working electrode (as described in the accompanying FIG. 6b)).

### Experimental Section

### Cell-Free Transcription Reaction

All transcription reactions were carried out in a 20 μL solution using 10 U/μl of T7-RNAP (NxGen T7 RNA polymerase (Biosearch, USA)), 1 U/μL Of RNAsi inhibitor (NxGen RNAse Inhibitor (Biosearch, USA)), 10 mM of each NTP (Jena Bioscience GmbH, Germany), and 100 nM of the inactive gene circuit (as described in the accompanying FIGS. 2A), 2b)). The solution was incubated at 37° C. for 2 hours to permit the transcription reaction. For the electrochemical experiments, a portion of the transcription reaction solution (1 μL or 10 μL) was placed onto the screen-printed silver electrode containing 100 μL of 50 mM Na₂HPO₄, 150 mM NaCl, pH 7.0, and the electrochemical signal was measured after 2 hours (as described in the accompanying FIG. 2c)).

### Modification of the Screen-Printed Electrodes With the Redox Probe

The redox probe (100 μM) was reduced for 1 hour in a solution of 0.4 mM TCEP (tris(2-carboxyethyl)phosphine hydrochloride) prepared in a buffer solution containing 150 mM NaCl and 50 mM NaH₂PO₄, PH 7. 0, to allow the reduction of the disulfide bonds. This solution was then diluted to the final concentration of 100 nM in the same buffer. The redox probe (20 μL) was then placed onto the silver working electrode (WE). After 1 hour incubation, the screen-printed electrode was washed with distilled water to remove the excess unbound DNA and subsequently, 20 μL of 3 mM mercaptohexanol (prepared in 150 mM NaCl, 50 mM NaH₂PO₄, PH 7.0) were placed onto the working electrode to passivate the electrode's surface. After 1 hour and 30 minutes of incubation, the screen-printed electrode was washed with distilled water.

### Electrochemical Experiments

All electrochemical measurements were taken at room temperature by using an MUX8-R2 multiplexer potentiostat (Palmsens Instruments, the Netherlands). The gene circuit was left to react in a test tube for 2 hours at 37° C., and then a portion thereof was transferred onto the surface of the screen-printed electrode as previously described. The experimental data were obtained by square-wave voltammetry in the potential range from −0.1 to −0.4 V with an increment of 0.001 V vs. Ag/AgCl, an amplitude of 10 mV and a frequency of 50 Hz, using the PSTrace 5.7v software (by Palmsens Instrument). All the experiments were carried out in 100 μL of buffering solution containing 50 mM Na₂HPO₄, 150 mM NaCl, PH 7.0 at 25° C.

### Advantages of the Present Invention

The method the present invention is based, therefore, on the development of a rapid, specific and highly sensitive test for the detection of antibodies and other analytes in complex sample arrays through the activation, induced by target antibodies, of a suitable gene circuit. The method requires the use only of those biological components which are necessary for the transcription process alone (not the translation process) in order to produce a specific final output RNA sequence. The advantages provided by the method of the present invention include, among the others, highly sensitive detection and quantification of antibodies. Moreover, the method does not require sophisticated instrumentation and provides results that can be interpreted by means of handy, low-cost electrochemical assays. The method of the present invention may also be useful for developing a platform for simultaneous and orthogonal detection of different target antibodies (multiplex). These advantages make the present method suitable for the execution of low-cost rapid tests at the point-of-care.

In comparison with the present inventors'known cell-free transcriptional switch described above, the method of the present invention envisages not only a different mechanism of activation of the gene circuit, but also a different method of detection. In the present disclosure, since the electrochemical signal is promoted by the specific hybridization of the transcribed RNA with the redox probe present on the surface of the screen-printed electrode (as illustrated in accompanying FIG. 1d)), detection is faster than with optical methods, because the latter are limited by the folding speed of the aptamer and by the time necessary for the latter to bind to its dye (TO-1). In addition, due to the possibility of miniaturization of the electrode surface, to the high programmability of the RNA-DNA interactions, and the orthogonality of the gene circuits, this approach supports the simultaneous detection of multiple antibodies. This approach does not require the transcriptional switch to have a complex design to permit a conformational change of the gene circuit, but is based simply on the co-location induced by the formation of the bond between the target antibody and two antigen-conjugated DNA strands.

Nevertheless, the method is not obvious because it requires appropriate selection and optimization of the antigen-conjugated strands to prevent them from activating the transcription in the absence of the target antibody.

### Exemplary Variants of the Present Invention

The application variants briefly described below are meant to illustrate, merely by way of non-limiting example, the broad scope of the present invention. Anyway, other variants may also be conceived by the skilled person on the basis of his knowledge in the art and the present description, which should all be considered to be included therein or easily derivable therefrom.

In fact, the method of the present invention can be implemented for the detection of a wide range of target molecules in complex sample arrays. The transcription reaction can be activated by the formation of the bond of any antibody for which the recognition element (also by using antibodies as recognition elements) can be conjugated to synthetic oligonucleotides.

Moreover, the method can also be implemented for the detection of small molecules by designing competitive assays (e. g., similar to those used in ELISA tests).

In addition, the method can also be implemented for the detection of clinically relevant proteins that recognize specific DNA or RNA sequences as transcription factors or DNA repair enzymes (such as, e. g. UDG (uracil-DNA glicosylase), Fpg (Formamidopyrimidine-DNA glycolyase).

Moreover, the method can also be implemented for the detection of nucleic acids by designing gene circuits capable of co-locating the two input strands.

The method can also be implemented for the detection of molecular analytes (such as, e.g., metal ions, small organic and inorganic molecules) recognized by the aptamers.

Other possible applications, apart from sensors, beside those described above rely on the fact that the transcription reaction controlled by a specific target analyte can be used to induce the production of a drug in the presence of a specific biomarker that may, for example, have therapeutic uses.

### Industrial Applicability

The present invention has made it possible to carry out a method for the detection of specific antibodies, proteins and small molecules in complex sample arrays, based on the activation of the in-vitro transcription, induced by the target, of a specific final (output) RNA sequence, as well as a related cell-free electrochemical biosensor.

### List of Cited Literature

[1] K. Pardee, A. A. Green, T. Ferrante, D. E. Cameron, A. DaleyKeyser, P. Yin, J. J. Collins, Cell 2014, 159, 940-954.
[2] K. Pardee, A. A. Green, M. K. Takahashi, D. Braff, G. Lambert, J. W. Lee, T. Ferrante, D. Ma, N. Donghia, M. Fan, N. M. Daringer, I. Bosch, D. M. Dudley, D. H. O'Connor, L. Gehrke, J. J. Collins, Cell 2016, 165, 1255-1266.
[3] J. K. Jung, K. K. Alam, M. S. Verosloff, D. A. Capdevila, M. Desmau, P. R. Clauer, J. W. Lee, P. Q. Nguyen, P. A. Pasten, S. J. Matiasek, J. F. Gaillard, D. P. Giedroc, J. J. Collins, J. B. Lucks, Nat Biotechnol 2020, 38, 1451-1459.
[4] A. D. Silverman, U. Akova, K. K. Alam, M. C. Jewett, J. B. Lucks, ACS Synth Biol 2020, 9, 671-677.
[5] S. Slomovic, K. Pardee, J. J. Collins, PNAS 2015, 112, 14429-14435.
[6] A. Patiño Diaz, S. Bracaglia, S. Ranallo, T. Patiño, A. Porchetta, F. Ricci, Programmable Cell-Free Transcriptional Switches for Antibodies Detection, ChemRxiv, 2021. DOI: 10.26434/chemrxiv-2021-swkv7
[7] P. Sadat Mousavi, S. J. Smith, J. B. Chen, M. Karlikow, A. Tinafar, C. Robinson, W. Liu, D. Ma, A. A. Green, S. O. Kelley, K. Pardee, Nat Chem 2020, 12, 48-55.
[8] E. Amalfitano, M. Karlikow, M. Norouzi, K. Jaenes, S. Cicek, F. Masum, P. Sadat Mousavi, Y. Guo, L. Tang, A. Sydor, D. Ma, J. D. Pearson, D. Trcka, M. Pinette, A. Ambagala, S. Babiuk, B. Pickering, J. Wrana, R. Bremner, T. Mazzulli, D. Sinton, J. H. Brumell, A. A. Green, K. Pardee, Nat Commun 2021, 12, 724.

1. A method for the electrochemical detection, with high sensitivity and specificity, of target molecular analytes directly in complex sample arrays, said method comprising:
the activation of an initially incomplete, i.e., inactive, DNA gene circuit, induced by a target analyte, wherein said analyte is selected from the group comprising, or consisting of, antibodies, proteins, small molecules, nucleic acids, derivatives thereof;
the transcription of a final RNA sequence by said activated DNA gene circuit;
the detection of said transcribed RNA sequence by means of an electrochemical biosensor.

2. The method according to claim 1, wherein said molecular analytes are selected from antibodies.

3. The method according to claim 1, wherein said complex sample arrays are selected from the group comprising, or consisting of, plasma, serum, blood, saliva, sweat, and the like.

4. The method according to claim 1, wherein said activation of said gene circuit is carried out by forming a bimolecular complex between it and a pair of antigen-conjugated DNA strands following the formation of a bivalent bond between the target analyte (preferably, the antibody) and the two antigens.

5. The method according to claim 1, wherein said transcription of a final RNA sequence is carried out by means of a RNA polymerase and nucleotides.

6. The method according to claim 1, wherein said electrochemical biosensor is constituted by a screen-printed silver electrode on which a redox probe complementary to the transcribed RNA sequence has been previously immobilized.

7. (canceled)

8. (canceled)
